(19) United States
(12) Patent Application Publication
(10) Pub. No.: US 2015/0265154 A1
(43) Pub. Date: Sep. 24, 2015

(54) SYSTEMS AND METHODS FOR A MULTI-ELEMENT MEDICAL SENSOR

(71) Applicant: COVIDIEN LP, MANSFIELD, MA (US)

(72) Inventors: Qiaojian Huang, Rocky Hill, CT (US); Youzhi Li, Longmont, CO (US); Charles Keith Haisley, Boulder, CO (US); Sarah Lynne Hayman, Boulder, CO (US); Friso Schlottau, Lyons, CO (US); Darshan Iyer, Superior, CO (US)

(21) Appl. No.: 14/632,945

(22) Filed: Feb. 26, 2015

Related U.S. Application Data
(60) Provisional application No. 61/946,144, filed on Feb. 28, 2014.

(51) Int. Cl. A61B 5/00; A61B 5/02

(52) U.S. Cl. CPC A61B 5/0095; A61B 5/02028; A61B 5/489; A61B 5/7203

(57) ABSTRACT

Various methods and systems for the use of multi-element photoacoustic sensors within medical devices configured for photoacoustic spectroscopy techniques are provided. The photoacoustic sensor includes two or more acoustic detectors spatially configured to increase the probability of a clinician properly placing at least one of the acoustic detectors on the measurement site over the blood vessels of interest. Further, the photoacoustic sensor includes a light delivery system configured to provide multiple light sources to the measurement site, such that each acoustic detector has an adequate light supply within close proximity. The present techniques additionally provide methods for processing each acoustic signal measured at the two or more acoustic detectors to calculate one or more physiological parameters of interest, such as cardiac output.

### CROSS-REFERENCE TO RELATED APPLICATION

This application claims the benefit of U.S. Provisional Application No. 61/946,144, entitled “SYSTEMS AND METHODS FOR A MULTI-ELEMENT MEDICAL SENSOR,” filed Feb. 28, 2014, which is herein incorporated by reference in its entirety.

### BACKGROUND

The present disclosure relates generally to medical devices and, more particularly, to systems and methods for a multi-element medical sensor for use in photoacoustic spectroscopy.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring patient characteristics. Such devices provide doctors and other healthcare personnel with the information they need to provide healthcare for their patients. As a result, such monitoring devices have become an indispensable part of modern medicine. For example, clinicians may wish to monitor a patient's blood flow to assess cardiac function. In particular, clinicians may wish to monitor a patient's cardiac output. The determination of cardiac output may provide information useful for the diagnosis and treatment of various disease states or patient abnormalities. For example, in cases of pulmonary hypertension, a clinical response may include a decrease in cardiac output.

Accordingly, there are a variety of clinical techniques that may be used for analyzing cardiac output. In one technique, an indicator, such as a dye or saline solution, is injected into a circulatory system of a patient, and information about certain hemodynamic parameters may be determined by assessing the dilution of the indicator after mixing with the bloodstream. For example, for patients with an indicator solution injected into a vein, photoacoustic monitoring techniques may be used to measure dilution of the indicator in a downstream artery after mixing in the blood. The extent of dilution relates to cardiac output and other hemodynamic parameters.

Such photoacoustic monitoring techniques may involve a photoacoustic sensor, an associated monitoring system, and/or associated methods used in conjunction with such sensors and/or systems. In one approach, the photoacoustic sensor utilizes light directed into a patient's tissue to generate acoustic waves that may be detected and analyzed to determine localized physiological parameter information. In particular, the light energy directed into the tissue may be provided at wavelengths that correspond to the absorption profile of tissue constituents of interest and/or to the absorption profile of the indicator of interest. The light absorbed by the material of interest results in a proportionate increase in kinetic energy, which results in the generation of acoustic waves. The acoustic waves may be detected and used to determine the amount of light absorption, and thus the quantity of the constituent of interest, in the illuminated region. For example, when an indicator is injected into a vein in a cardiovascular system, a diluted temporal profile of the indicator may be measured via a photoacoustic monitoring technique in a downstream artery. Algorithms applied to photoacoustic indicator dilution curves may be used to estimate hemodynamic properties, such as cardiac output. Photoacoustic monitoring may also be used to assess other physiological parameters.

In many systems, the photoacoustic sensor may be placed on a measurement site above the patient's targeted vessels, such as on a superficial temporal artery, so that the light energy is efficiently delivered and the acoustic waves are efficiently detected. Unfortunately, it may be difficult for clinicians to properly place the photoacoustic sensor on the measurement site due to variations between different individuals in the size, the shape, or the location of the targeted vessels. For example, the measurement site over the superficial temporal artery may be difficult to find because the artery may be relatively small or because the artery may be positioned in different locations for different individuals. Accordingly, there is a need for a photoacoustic sensor having greater position tolerance, such that clinicians are able to easily apply the photoacoustic sensor to the patient without making adjustments. Further, there is a need for a photoacoustic sensor that provides greater position tolerance without compromising the accuracy of the measured acoustic waves and physiological parameters.

### BRIEF DESCRIPTION OF THE DRAWINGS

Advantages of the disclosed techniques may become apparent upon reading the following detailed description and upon reference to the drawings in which:

FIG. 1 is a block diagram of a patient monitor and a photoacoustic sensor in accordance with an embodiment, where the photoacoustic sensor includes one or more acoustic detectors and a plurality of light sources disposed within an element housing;

FIG. 2 is a block diagram of the element housing of FIG. 1 having three acoustic detectors and three light sources in a triangular arrangement, where the arrangement is configured to provide greater horizontal and vertical position tolerance for the placement of the photoacoustic sensor;

FIG. 3 illustrates the triangular arrangement of FIG. 2 within a compact element housing, where the arrangement includes light sources disposed external to the acoustic detectors, and where the compact element housing is configured to distinguish between multiple blood vessels to identify the target blood vessel;

FIG. 4 illustrates the triangular arrangement of FIG. 2, where each acoustic detector and/or each light source of the arrangement have different dimensions and/or size ratios;

FIG. 5 illustrates an embodiment of the one or more acoustic detectors and the plurality of light sources disposed on the photoacoustic sensor of FIG. 1, where the having acoustic detectors and the plurality of light sources are disposed in an array;

FIG. 6 is a block diagram of an embodiment of the photoacoustic monitoring system of FIG. 1 applied to a measurement site having a blood vessel of interest, in accordance with an embodiment of the present disclosure;

FIG. 7 is an isometric view of an embodiment of a light splitting structure configured to house the components of the photoacoustic sensor of FIG. 6;

FIG. 8 is a front view of an embodiment of an integrated prism light splitting structure configured to house the components of the photoacoustic sensor of FIG. 6;

FIG. 9 is a side view of an embodiment of a stepped-prism light splitting structure configured to house the components of the photoacoustic sensor of FIG. 6;

FIG. 10A is an embodiment of a multi-fiber light splitting structure configured to receive a plurality of optical fibers and to house the components of the photoacoustic sensor of FIG. 6;

FIG. 10B is an embodiment of a prism subassembly of the multi-fiber light splitting structure of FIG. 10A illustrating two prisms separated at the hypotenuse surface with an air gap;

FIG. 11 is a flow chart for calculating cardiac output based on one or more denoised indicator dilution (ID) curves generated from the acoustic signals and/or the optical signals provided by the sensor 12 of FIGS. 1-10A;

FIG. 12 is a flow chart for selecting relevant independent component(s) among the plurality of independent components generated for each raw ID curve, where each raw ID curve corresponds to the acoustic signal and/or the optical signal of FIG. 11; and

FIG. 13 is a flow chart for calculating the final cardiac output based on the one or more denoised ID curves of FIG. 11.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Provided herein are systems and methods for a multi-element photoacoustic sensor utilized in photoacoustic spectroscopy techniques. As described in detail below, the photoacoustic sensor includes two or more acoustic detectors spatially positioned to increase the probability of a clinician properly placing at least one of the acoustic detectors on the measurement site over the blood vessels of interest. Further, the photoacoustic sensor includes a light delivery system configured to provide multiple light sources to the measurement site, such that each acoustic detector has an adequate light supply within close proximity. In particular, the light delivery system includes techniques for splitting a single light source into a plurality of distinct light sources that are provided normal to the measurement site. In certain embodiments, the present techniques additionally provide methods for processing each acoustic signal measured at the two or more acoustic detectors to calculate one or more hemodynamic properties of interest, such as cardiac output.

Specifically, the photoacoustic system may include a photoacoustic sensor having sensor components or elements that are spatially configured to increase position tolerance. For example, the photoacoustic sensor may be placed at a measurement site on a subject, such as on the wrist, the palm, the neck, the forehead, the temple, and/or anywhere that a blood vessel (e.g., arteries and/or veins) are accessible noninvasively. In certain situations, the photoacoustic sensor may be improperly placed near the measurement site, and may require additional adjustments (e.g., repositioned horizontally and/or vertically) so that the sensor elements are properly positioned over the measurement site. Accordingly, the photoacoustic sensor provided herein includes multiple sensor elements that allow the sensor to be placed on the measurement site without additional adjustments. In particular, the photoacoustic sensor may use any suitable light source elements (e.g., fiber optics, laser, laser diode, etc.) to deliver light to the measurement site on the subject, and acoustic detector elements (e.g., ultrasound detectors) to sense the pressure response of the tissue induced by the absorption of light. For example, three acoustic detectors may be arranged in a linear array within a rectangular housing on the sensor, having a light source through the center of each acoustic detector. In other embodiments, 2, 4, 5, 6, 7, 8, 9, 10, 11, 12, 13, 14, 15 or more acoustic detectors may be arranged in a variety of configurations (e.g., a one or two dimensional array, a plus-sign, a triangle, etc.) within various shapes of housing (e.g., circle, square, etc.), having a light source through the center of each acoustic detector or positioned outside near the acoustic detector.

In certain embodiments, the photoacoustic sensor includes a light delivery system configured to split a single light source into a plurality of light sources, such that each acoustic detector has an adequate light supply within a close proximity. In particular, the light delivery system may be configured to bend or angle each of the plurality of light sources, so that light is delivered normal to the measurement site. For example, the light delivery system may include a single mode optical fiber coupled to a diffractive optical element (DOE). In certain embodiments, the DOE is configured to receive the single beam of light and split the single beam into multiple beams of light. The resulting beams expand and separate as they pass through an optical travel element within the light delivery system. Further, a prism within the light delivery system may be configured to bend the resulting beams of light 90 degrees, such that they are normal to the measurement site. Accordingly, the light delivery system is configured to provide light sources normal to the measurement site for each acoustic detector, such that each acoustic detector has at least one light source near or within a close proximity.

The photoacoustic system may measure the pressure response that is received at each acoustic detector, and may process the received information to calculate one or more hemodynamic properties of interest. In particular, each acoustic detector on the sensor may receive the pressure response as an acoustic pressure signal representing pressure versus time. The acoustic pressure signal may be further processed to derive the photoacoustic signal, which in turn may be used to calculate any of a number of physiological parameters of interest, including cardiac output. In certain situations, it may be desirable to apply an independent component analysis technique to the photoacoustic signals to remove undesired noise (e.g., heart rate, cardiac noise, electronic crosstalk, etc.) and to determine a denoised photoacoustic signal. Further, in certain embodiments, each denoised photoacoustic signal may be used to calculate cardiac output, and the final cardiac output may be determined by averaging the cardiac output calculated for each denoised photoacoustic signal. In other embodiments, particular denoised photoacoustic signals may be selected based on various parameters (e.g., signal quality, signal strength, etc.), and the final cardiac output may be determined based on the selected denoised photoacoustic signal.

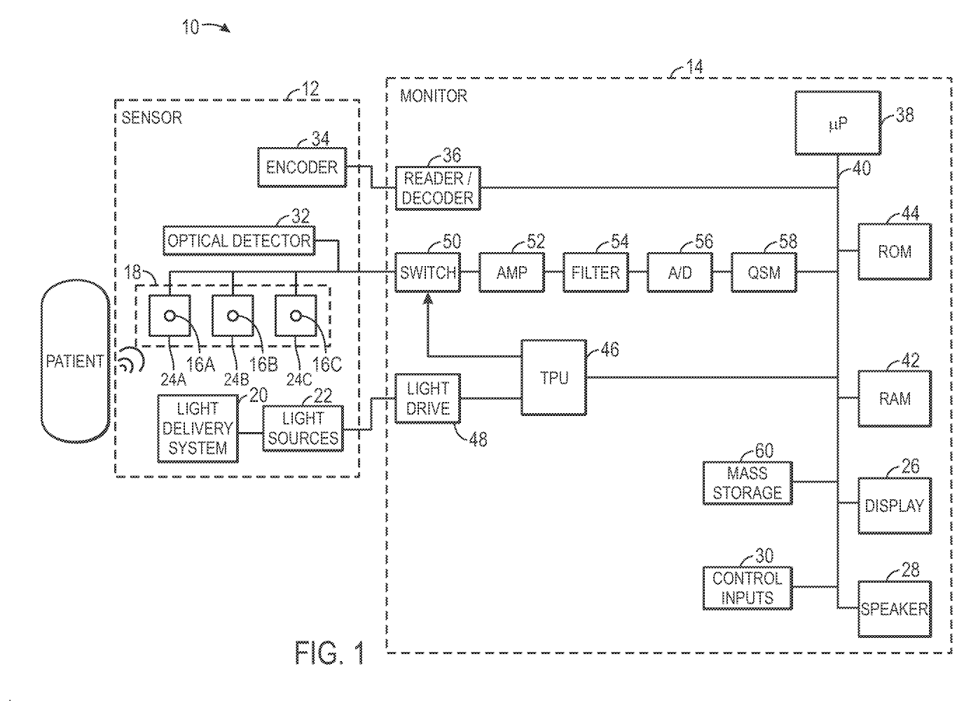

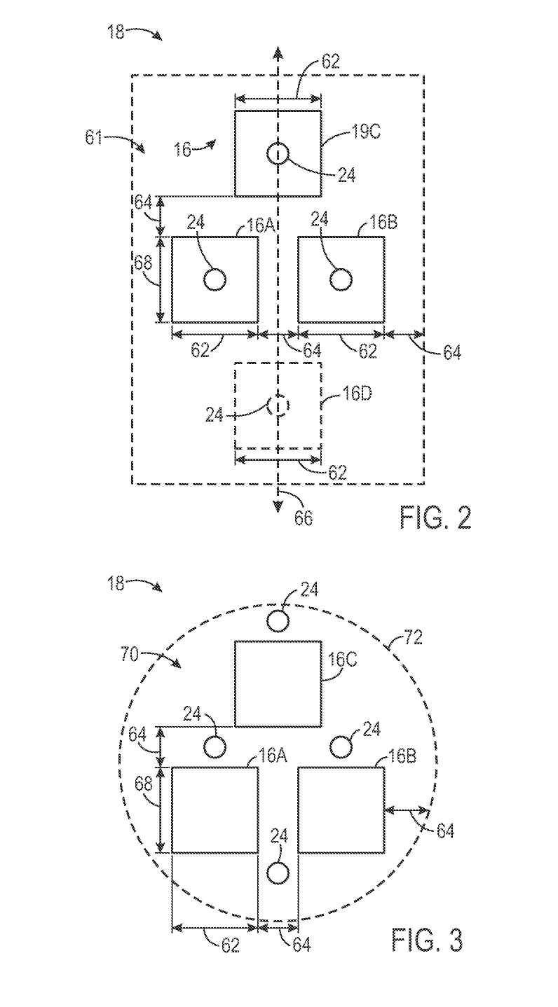

With the forgoing in mind, FIG. 1 depicts a block diagram example of a photoacoustic monitoring system 10 that may be utilized in determining one or more hemodynamic properties of interest, such as cardiac output. The system 10 includes a photoacoustic sensor 12 and a monitor 14. In particular, the photoacoustic sensor 12 may include one or more acoustic detectors 16 arranged in a linear array. In some embodiments, the acoustic detectors 16 are arranged within an element housing 18 disposed on the photoacoustic sensor 12 (e.g., disposed on a sensor body and/or disposed on a patient-contacting surface of the sensor 12). Further, the sensor 12 may include a light delivery system 20 configured to split one or more light sources 22 into a plurality of light emitting components 24, such that each acoustic detector 16 may be associated with a light emitting component 24 located near or within a proximate distance. For example, in the illustrated embodiment, each light emitting component 24 is disposed approximately in the center of each acoustic detector 16 within the element housing 18. In should be noted that in other embodiments, the one or more acoustic detectors 16 and the plurality of light emitting components 24 may be arranged in other configurations within the element housing 18, as described in detail with respect to FIGS. 2-5.

Some photoacoustic systems 10 may include one or more photoacoustic sensors 12 to generate physiological signals for different regions of a patient. For example, in certain embodiments, one sensor 12 may have sufficient penetration depth to generate physiological signals from deep vessels (e.g., pulmonary artery and/or pulmonary vein), while in other embodiments, more than one sensor 12 (e.g., two or more sensors) may be used to monitor physiological parameters. In certain embodiments, an array of element housings 18 disposed on the sensor 12, with each element housing 18 having one or more acoustic detectors 16 and a plurality of light emitting components 24, may be utilized on a measurement site to generate physiological signals over a larger measurement region, as described in detail with respect to FIG. 5. In addition, the system 10 may be used in conjunction with other types of medical sensors, e.g., pulse oximetry or regional saturation sensors, to provide input to a multiparameter monitor. In certain embodiments, the sensor 12 also includes an optical detector 32 that may be a photodetector, such as a silicon photodiode package, selected to receive light in the range emitted from the plurality of light emitting components 24. In the present context, the optical detector 32 may be referred to as a detector, a photodetector, a detector device, a detector assembly or a detector component. Further, the detector 32 and the plurality of light emitting components 24 may be referred to as optical components or devices.

The sensor 14 may emit light at certain wavelengths into a subject's tissue and may detect acoustic waves (e.g., ultrasound waves) generated in response to the emitted light. The monitor 14 may be capable of calculating physiological characteristics based on signals received from the sensor 12 that correspond to the detected acoustic waves. The monitor 14 may include a display 26 and/or a speaker 28 which may be used to convey information about the calculated physiological characteristics to a user. Further, the monitor 14 may be configured to receive user inputs via control input circuitry 30. The sensor 12 may be communicatively coupled to the monitor 14 via a cable or, in some embodiments, via a wireless communication link. For example, the monitor 14 may include one or more sensor inputs utilized to communicate with the sensor 12.

As noted above, in certain embodiments, the sensor 12 may include one or more acoustic detectors 16 (e.g., ultrasound transducers), and the light source 22 that is split into a plurality of light emitting components 24 via the light delivery system 20. The disclosed embodiments may generally describe the use of continuous wave (CW) light sources to facilitate explanation. However, it should be appreciated that the photoacoustic sensor 12 may also be adapted for use with other types of light sources, such as pulsed light sources, in other embodiments. In certain embodiments, the light source 22 may be associated with one or more optical fibers for conveying light from one or more light generating components to the light delivery system 20. Specifically, the light source 22 may be associated with a single mode optical fiber (e.g., unimode fiber, monomode optical fiber, etc.) configured to provide a single mode of light traveling in a single defined path. The single mode fiber may have an optical core between approximately 8 microns and 10 microns. The single mode optical fiber may be configured to provide a single ray of light as the light source 22, which in certain embodiments be split into the plurality of light emitting components 24 via the light delivery system 20, as described in detail below. It should be noted that in other embodiments, the light source 22 may be associated with other types of optical fibers, such as multi-mode optical fibers and/or special purpose optical fibers. In such embodiments, one or more single light sources 22 may be provided directly to the one or more acoustic detectors 16.

In certain embodiments, the light source 22 may be associated with one, two, or more light emitting components (such as light emitting diodes) adapted to transmit light at one or more specified wavelengths. For example, the light source 22 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1100 nm or between about 600 nm to about 900 nm) that is absorbed by a constituent of interest in the blood or tissue. For example, wavelengths between about 500 nm to about 600 nm, corresponding with green visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1100 nm) may be used. In one embodiment, the selected wavelengths of light may penetrate between 1 mm to 3 cm into the tissue of the patient. In certain embodiments, the selected wavelengths may penetrate through bone (e.g., the rib cage) of the patient.

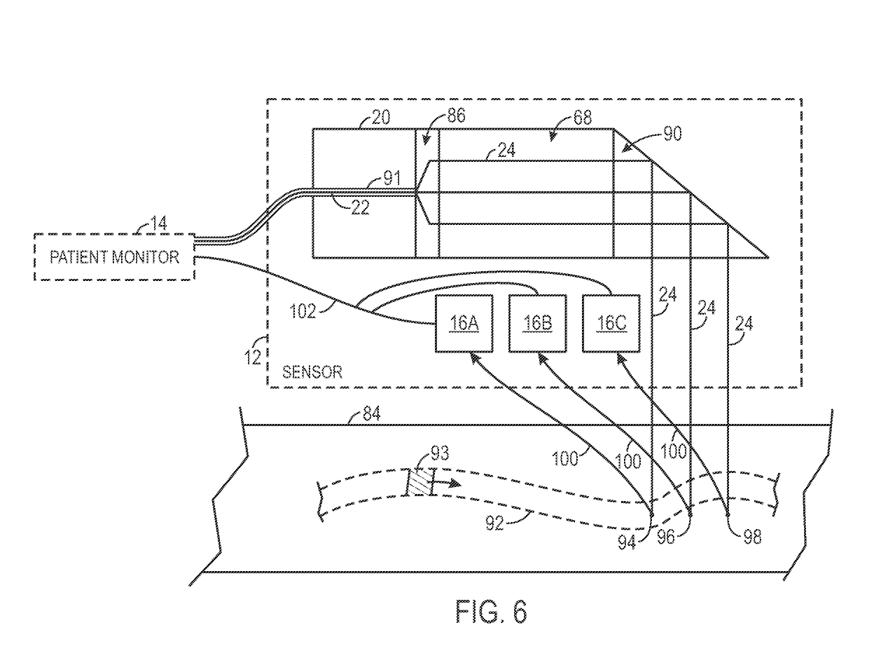

In particular, the light delivery system 20 may be configured to receive the single mode of light (e.g., single ray, single beam, etc.) transmitted by the light source 22, split the single ray of light into a plurality of light rays, and bend the plurality of light rays so that they arrive normal to the measurement site on the subject as a plurality of light emitting components 24, as discussed in further detail with respect to FIG. 6. In this manner, the light delivery system 20 provides a plurality of light emitting components 24 for the one or more acoustic detectors 16 without introducing additional wiring (e.g., optical fibers) between the sensor 12 and the monitor 14. It should be noted that while the illustrated embodiment depicts three split light emitting components 24, in other embodiments, the light delivery system 20 may be configured to split the single mode of light from the light source 22 into 2, 4, 5, 6, 7, 8, 9, 10, 11, 12, 13, 14, 15, or more light emitting components 24 to provide sufficient light for the one or more acoustic detectors 16. Further, the light delivery system 20 may be configured to bend the plurality of light emitting components 24 by approximately 90 degrees, such that the light is supplied approximately normal to the surface of the measurement site of the subject. In this manner, the light delivery system 20 may be configured to maximize the amount of light provided to the measurement site. However, it should be noted that in other embodiments, each of the plurality of light emitting components 24 may be associated with one or more optical fibers for conveying light from one or more light generating components directly to the tissue measurement site.

In certain embodiments, the plurality of light emitting components 24 may be associated with one or more optical fibers and/or one or more light channels (e.g., light pipe, light holes, etc.) having a reflective inner coating. For example, the plurality of light rays generated by the light delivery system 20 may be provided normal to the measurement site near and/or within close proximity of the acoustic detectors 16 within the element housing 18. Providing the plurality of light rays close to the acoustic detectors 16 may improve the efficiency with which light is provided to the measurement site and with which the acoustic waves are detected from the measurement site. In certain embodiments, a plurality of short optical fibers may be used to transmit the plurality of light rays from the light delivery system 20 to within the element housing 18. For example, holes formed through approximately the center of each acoustic detector 16 may be used for the placement of each optical fiber from the light delivery system 20, as further described with respect to FIGS. 2, 4, and 5. As a further example, holes may be formed outside, but near and/or within close proximity of the acoustic detector 16, and may be used for the placement of the plurality of optical fibers from the light delivery system 20, as further described with respect to FIG. 3. In other embodiments, one or more light channels (e.g., light pipe, light holes, etc.) having a reflective inner coating may be used in lieu of the plurality of optical fibers to transmit the plurality of light rays from the light delivery system 20. In such embodiments, the light channels may be configured to provide the plurality of light rays normal to the measurement site directly from the light delivery system 20.

In one example, the one or more acoustic detectors 16 may be one or more ultrasound transducers, such as a focused ultrasound transducer, suitable for detecting ultrasound waves emanating from the tissue in response to the emitted light and for generating a respective optical or electrical signal in response to the ultrasound waves. For example, the acoustic detectors 16 may be suitable for measuring the frequency and/or amplitude of the acoustic waves, the shape of the acoustic waves, and/or the time delay associated with the acoustic waves with respect to the light emission that generated the respective waves. In one embodiment, the acoustic detector 16 may be an ultrasound transducer employing piezoelectric or capacitive elements to generate an electrical signal in response to acoustic energy emanating from the tissue of the patient or subject, i.e., the transducer converts the acoustic energy into an electrical signal.

In the illustrated embodiment, the three acoustic detectors 16 are approximately are arranged in a linear array within the element housing 18. For example, each acoustic detector 16 is approximately 3 mm in width and 5 mm in length, and may be positioned with an approximately 0.5 mm gap between adjacent acoustic detectors 16 (e.g., separation gap). In other embodiments and configurations (as described with respect to FIGS. 2-5), the acoustic detectors 16 may be a variety of dimensions, and may be configured in a variety of arrangements. In certain embodiments, in lieu of the one or more acoustic detectors 16, a single acoustic detector 16 may be provided (not shown). In such embodiments, the acoustic detector 16 may have a greater surface area, such as for example, a surface area approximately equivalent to the surface area provided by two or more individual acoustic detectors 16. For example, the single acoustic detector 16 may have approximately the same surface of 1, 2, 3, or more single acoustic detectors 16, and may include three or more optical fibers and/or channels for the placement of three or more light emitting components 24. Further, during operation of the single acoustic detector 16, the measurement site of the subject may include light and/or acoustic barriers (e.g., black strips) configured to separate the measurement site into a plurality of measurement sites.

The disclosed embodiments depict reflectance-type sensor arrangements in which the plurality of light emitting components 24, the acoustic detectors 16, and a light detector 32 are on the same side of the sensor 12. It should be understood that transmission-type arrangements are also contemplated in which the plurality of light emitting components 24, the acoustic detectors 16, and the light detector 32 are on opposing sides of a tissue when applied to a patient (e.g., light emitting components 24 on a first side, the detectors 16, 32 on a second side). For a transmission mode sensor and a reflectance-type sensor, the optical signal and the photoacoustic signal may have a negative correlation between these signals.

The photoacoustic sensor 12 may include a memory or other data encoding component, depicted in FIG. 1 as an encoder 34. For example, the encoder 34 may be a solid state memory, a resistor, or combination of resistors and/or memory components that may be read or decoded by the monitor 12, such as via reader/decoder 36, to provide the monitor 14 with information about the attached sensor 12. For example, the encoder 34 may encode information about the sensor 12 or its components (such as information about the plurality of light emitting components 24 and/or the one or more acoustic detectors 16). Such encoded information may include information about the configuration or location of photoacoustic sensor 12, information about the type of lights source(s) 22 or 24 present on the sensor 12, information about the wavelengths, light wave frequencies, chirp durations, and/or light wave energies which the light source(s) 22 or 24 are capable of emitting and the properties and/or detection range of the optical detector 32, information about the nature of the acoustic detectors 16, and so forth. This information may allow the monitor 14 to select appropriate algorithms and/or calibration coefficients for calculating the patient's physiological characteristics, such as the amount or concentration of a constituent of interest in a localized region, such as a blood vessel.

In one implementation, signals from each of the acoustic detectors 16 (and decoded data from the encoder 34, if present) and the optical detector 32 may be transmitted to the monitor 14 through one or more sensor inputs on the monitor 14. The monitor 14 may include data processing circuitry (such as one or more processors 38, application specific integrated circuits (ASICS), or so forth) coupled to an internal bus 40. Also connected to the bus 40 may be a RAM memory 42, a ROM memory 44, a speaker 28 and/or a display 26. In one embodiment, a time processing unit (TPU) 46 may provide timing control signals to light drive circuitry 48, which controls operation of the light source 22, such as to control when, for how long, and/or how frequently the light source 22 is activated. Accordingly, the light drive circuitry 48 may also be responsible for controlling the operation of the plurality of light emitting components 24, including how frequently the plurality of light emitting components 24 are activated, which light sources among the plurality of light emitting components 24 are activated, and the multiplexed timing for the different light sources.

The TPU 46 may also control or contribute to the operation of the acoustic detectors 16 and/or the optical detector 32 such that timing information for data acquired using the acoustic detectors 16 and/or the optical detector 32 may be obtained. Such timing information may be used in interpreting the acoustic wave data and/or in generating physiological information of interest from such acoustic data. For example, the timing of the acoustic data acquired using the acoustic detectors 16 may be associated with the light emission profile of the plurality of light emitting components 24 during data acquisition. Likewise, in one embodiment, data acquisition by the acoustic detectors 16 may be gated, such as via a switching circuit 50, to account for differing aspects of light emission. For example, operation of the switching circuit 50 may allow for separate or discrete acquisition of data that corresponds to different respective wavelengths of light emitted at different times. Similarly, the data acquired from the optical detector 32 may be gated via the switched circuit 50. Further, while the disclosed block diagram shows the signal from each acoustic detector 16 being supplied to the same path, it should be understood that each acoustic detector 16 may be gated via the switched circuit 50. Accordingly, operation of the switching circuit 50 may also allow for separate or discrete acquisition of data that corresponds to a particular acoustic detector 16.

The received signal from the acoustic detectors 16 and/or the optical detector 32 may be amplified (such as via amplifier 52), may be filtered (such as via filter 54), and/or may be digitized if initially analog (such as via an analog-to-digital converter 56). The digital data may be provided directly to the processor 38, may be stored in the RAM 42, and/or may be stored in a queued serial module (QSM) 58 prior to being downloaded to RAM 42 as QSM 58 fills up. In one embodiment, there may be separate, parallel paths for separate amplifiers, filters, and/or A/D converters provided for different respective light wavelengths or spectra used to generate the acoustic data. Further, while the disclosed block diagram shows the signal from the optical detector 32 and the acoustic detectors 16 being supplied to the same path (e.g., a path that may include the switch 50, the amplifier 52, the filter 54, the A/D converter 56, and/or the QSM 58), it should be understood that these signals may be received and processed on separate paths or separate channels. For example, in certain embodiments, the signal received from each acoustic detector 16 may be supplied and processed along a separate path that may include the switch 50, the amplifier 52, the filter 54, the A/D converter 56, and/or the QSM 58. Accordingly, the data for each acoustic detector 16 may be provided directly to the processor 38 for additional processing and analysis, may be stored in the RAM 42, and/or may be stored in a queued serial module (QSM) 58 prior to being downloaded to RAM 42 as QSM 58 fills up.

The data processing circuitry, such as processor 38, may derive one or more physiological characteristics based on data generated by the photoacoustic sensor 12. For example, based at least in part upon data received from each acoustic detector 16, the processor 38 may calculate the amount or concentration of a constituent of interest in a localized region of tissue or blood using various algorithms. In one embodiment, the processor 38 may calculate one or more of various hemodynamic parameters, such as cardiac output, total blood volume, extravascular lung water, intrathoracic blood volume, and/or macro and microvascular blood flow from signals obtained from the sensor 12. In certain embodiments, these algorithms may use coefficients, which may be empirically determined, that relate the detected acoustic waves generated in response to emitted light waves at a particular wavelength or wavelengths to a given concentration or quantity of a constituent of interest within a localized region.

In one embodiment, the processor 38 may access and execute coded instructions, such as for implementing the algorithms discussed herein, from one or more storage components of the monitor 14, such as the RAM 42, the ROM 44, and/or a mass storage 60. Additionally, the RAM 42, the ROM 44, and/or the mass storage 60 may serve as data repositories for information such as templates for LFM reference chirps, coefficient curves, and so forth. For example, code encoding executable algorithms may be stored in the ROM 44 or mass storage device 60 (such as a magnetic or solid state hard drive or memory or an optical disk or memory) and accessed and operated according to processor 38 instructions using stored data. Such algorithms, when executed and provided with data from the sensor 12, may calculate one or more physiological characteristics as discussed herein (such as the type, concentration, and/or amount of an indicator). Once calculated, the physiological characteristics may be displayed on the display 26 for a caregiver to monitor or review. In certain embodiments, the calculated physiological characteristics, such as the hemodynamic parameters, may be sent to a multi-parameter monitor for further processing and display. Additionally or alternatively, the processor 38 may use the algorithms to calculate one or more cardiac outputs based on signals received from each acoustic detector 16, and/or calculate a final cardiac output based on an average of the one or more cardiac outputs or based on a signal received from a particular acoustic detector 16. The one or more cardiac output and/or the final cardiac output may be displayed on the display 26 of the monitor 14.

As noted above, in certain embodiments, the signal received from each acoustic detector 16 may be supplied and processed along a separate path or channel (e.g., each path or channel comprising the switch 50, the amplifier 52, the filter 54, the A/D converter 56, and/or the QSM 58) within the patient monitor 14. For example, in the illustrated embodiment, the three acoustic detectors 16 disposed on the sensor 12 may provide three photoacoustic signals (e.g., paths and/or channels of data) to the monitor 14 for processing. In some embodiments, the photoacoustic signal generated by a particular acoustic detector 16 may be influenced by the light provided by an adjacent detector 16. For example, the light emitted by a first light emitting component 24a may cause an acoustic effect detected by a first acoustic detector 16a and may also affect the photoacoustic signal generated by a second acoustic detector 16b, where the second acoustic detector 16b is adjacent to the first acoustic detector 16a. In certain embodiments, the contribution of the photoacoustic signal from the first light emitting component 24a may be approximately 10%, 20%, 25%, or more. Further, in certain embodiments, the light emitted by the first light emitting component 24a may have a negligible influence on a non-adjacent acoustic detector, such as a third acoustic detector 16c.

Accordingly, the monitor 14 may be configured to determine and/or select one or more light emitting components 24 out of a set of light emitting components 24 and/or one or more acoustic detectors 16 (e.g., one or more adjacent acoustic detectors 16, a group and/or set of acoustic detectors 16, a portion and/or a subset of the acoustic detectors 16, etc.) configured to provide the strongest and/or highest quality photoacoustic signals and/or the highest quality data. For example, the monitor 14 may be configured to determine and/or select one or more acoustic detectors 16 out of a plurality of acoustic detectors 16 that are disposed in the closest proximity to a portion of the patient's tissue over the patient's targeted vessels. Further, the monitor 14 may be configured to drive one or more light emitting components 24. In some embodiments, the detectors 16 selected by the monitor 14 may a signal quality above a predetermined threshold. In other embodiments, the monitor 14 may be configured to select all of the acoustic detectors 16, thereby utilizing all of the acoustic detectors 16 to determine one or more physiological parameters of interest. In another embodiment, the system 10 may cycle through the various emitter 24/detector 16 combinations to find a highest signal quality. Further, the system 10 may be configured to periodically check the signal quality of various configurations or may be configured to find a different active emitter 24/detector 16 configuration when the signal quality drops below a threshold. In one embodiment, the system 10 may be configured for a low power mode in which the smallest number of active emitters 24 providing a signal quality above a threshold is selected.

As such, the monitor 14 may be configured to select one or more acoustic detectors 16 out of a plurality of acoustic detectors 16 disposed on the sensor 12 to provide the sensor 12 with greater position tolerance, such that clinicians are able to easily apply the sensor 12 to the patient without making multiple adjustments. In some embodiments, the acoustic detectors 16 and the light emitting components 24 may be disposed on the sensor 12 in a manner that provides greater position tolerance. For example, the arrangements and/or configurations of the acoustic detectors 16 and the light emitting components 24 may be disposed in different shapes (e.g., triangular, square, arrays, etc.) and in different numbers (e.g., 1, 2, 3, 4, 5, 6, 7, 8, 9, 10, or more acoustic detectors 16), as described further with respect to FIGS. 2, 3, 4, and 5. Further, each acoustic detector 16 and light emitting component 24 may be different dimensions, sizes, and/or size ratios and may be disposed in different types of housing, as described further with respect to FIGS. 2, 3, 4, and 5.

FIG. 2 illustrates a two-dimensional arrangement 61 of the acoustic detectors 16 and the light emitting components 24 within the element housing 18, where the arrangement 61 is configured to provide greater tolerance in two dimensions (for example, such as both horizontal and vertical tolerance) as the sensor 12 is being placed on the subject. Specifically, the arrangement 61 includes a first acoustic detector 16a disposed adjacent to a second acoustic detector 16b. Further, the arrangement 61 includes a third acoustic detector 16c disposed above the first and second acoustic detectors 16a and 16b, forming a triangular arrangement or pattern 61. In certain embodiments, the arrangement 61 further includes a fourth acoustic detector 16d disposed below the first and second acoustic detectors 16a and 16b, forming a diamond arrangement. In particular, the third acoustic detector 16c and the fourth acoustic detector 16d may be configured to provide greater tolerance in a first direction (for example, vertically) during application of the sensor 12. For example, in certain situations, the photoacoustic sensor may be improperly placed near the measurement site, and may require additional adjustments (e.g., repositioned horizontally and/or vertically) so that the sensor elements (e.g., acoustic detectors 16 and/or the plurality of light emitting components 24) are properly positioned over the measurement site (e.g., above the target blood vessels). Accordingly, the first acoustic detector 16a and/or the second acoustic detector 16b may provide greater tolerance in a second direction different than the first direction (for example, horizontally), and may help reduce additional adjustments to the left and/or the right to reposition the sensor 12 above the target blood vessels. Likewise, the third acoustic detector 16c and/or the fourth acoustic detector 16d may provide greater vertical tolerance, and may help reduce additional adjustments to the top and/or the bottom to reposition the sensor 12 above the target blood vessels.

In certain embodiments, the arrangement 61 may be a triangular configuration having three acoustic detectors 16 with each acoustic detector 16 having the light emitting component 24 disposed approximately in the middle. For example, the first acoustic detector 16a may be disposed adjacent to the second acoustic detector 16b. Each acoustic detector 16 may be a first distance 62 in length, and may be separated by surrounding acoustic detectors 16 with a separation gap 64. Further, the third acoustic detector 16c may be disposed above the first acoustic detector 16a and the second acoustic detector 16b, such that the midpoint along the first distance 62 of the third acoustic detector 16c and the midpoint along the separation gap 64 between the first acoustic detector 16a and the second acoustic detector 16b lie along a center axis 66. In other embodiments, it should be noted that the third acoustic detector 16c may lie anywhere within the element housing 18 above the first acoustic detector 16a and the second acoustic detector 16b, such as for example, directly above the first acoustic detector 16a or directly above the second acoustic detector 16b. Further, in certain embodiments, the fourth acoustic detector 16d may be disposed below the first acoustic detector 16a and the second acoustic detector 16b, such that the midpoint along the first distance 62 of the fourth acoustic detector 16c and the midpoint along the separation gap 64 between the first acoustic detector 16a and the second acoustic detector 16b lie along the center axis 66. In other embodiments, the fourth acoustic detector 16d may lie anywhere within the element housing 18 below the first acoustic detector 16a and the second acoustic detector 16b, such as for example, directly below the first acoustic detector 16a or directly below the second acoustic detector 16b.

As noted above with respect to FIG. 1, the acoustic detectors 16 and the plurality of light emitting components 24 may be a variety of dimensions. For example, the first distance 62 may be approximately 1 mm, 2 mm, 3 mm, 4 mm, 5 mm, 6 mm, 7 mm, 8 mm or more in length. Further, the separation distance 64 (e.g., distance between any two acoustic detectors 16), may be 0.1 mm, 0.2 mm, 0.3 mm, 0.4 mm, 0.5 mm, 0.6 mm, 0.7 mm, or more. In addition, a second distance 68 for each acoustic detector 16 may be approximately 1 mm, 2 mm, 3 mm, 4 mm, 5 mm, 6 mm, 7 mm, 8 mm or more in width. As noted above, in certain embodiments, each of the plurality of light emitting components 24 may be disposed approximately in the center of each acoustic detector 16. For example, the plurality of light emitting components 24 may be associated with one or more optical fibers and/or one or more light channels (e.g., light pipe, light holes, etc.) having a reflective inner coating. In certain embodiments, each optical fiber may be disposed within a hole (e.g., opening, slot, etc.) approximately at the center of each acoustic detector 16. Specifically, each optical fiber may be between approximately 580 micrometers and 660 micrometers, and the hole or opening within the acoustic detector 16 configured to hold the optical fiber in place may be between approximately 610 micrometers and 690 micrometers. In certain embodiments, the one or more light channels with reflective inner coatings may be sized approximately similar to the dimensions of the optical fibers and/or the holes configured to hold the optical fibers. It should be noted that in other embodiments, each light emitting component 24 may be disposed external to the acoustic detectors 16, but still near and/or within a close proximity to the acoustic detectors 16, as further described with respect to FIG. 3.

FIG. 3 illustrates a two-dimensional arrangement 70 of the acoustic detectors 16 and the light emitting components 24 within a more compact circular element housing 72, where the compact element housing 72 is configured to distinguish between multiple blood vessels to identify the target blood vessel. Specifically, the arrangement 70 may be a triangular configuration having three acoustic detectors 16 and four or more light emitting components 24 disposed external to the acoustic detector 16 and near or within a close proximity to the acoustic detectors 16. In particular, the arrangement 70 may be disposed within the circular element housing 72, which may be more compact than the element housings 18 illustrated in FIGS. 1-2. In certain situations, the more compact element housing 72 may be configured to distinguish between multiple blood vessels that are positioned close to one another within and/or close to the measurement site. For example, in certain situations, the target blood vessel (e.g., superficial temporal artery on a subject's temple region) may be surrounded by other blood vessels. In such situations, the clinician may have difficult identifying the exact position to place the sensor 12 so that accurate measurements are received from the target blood vessel. Accordingly, the compact element housing 18 may be configured to focus the light emitted and the acoustic waves detected from a particular region of the measurement site. Further, the smaller size of the compact element housing 18 may provide greater placement options on the subject.

In particular, it should be noted that the number of light emitting components 24 may be greater than, less than, or the equivalent to the number of the acoustic detectors 16, such that each acoustic detector 16 is associated with one or more light emitting components 24 (e.g., ratio of light emitting components 24 to acoustic detectors 16 may be greater than 1:1). For example, the illustrated embodiment depicts three acoustic detectors 16 arranged in the triangular arrangement 16, and four light emitting components 24 disposed around the external perimeter of the acoustic detectors 16. In other embodiments, the element housing 18 may include 1, 2, 3, 5, 6, 7, 8, 9, 10, 11, 12, 13, 14, 15 or more light emitting components 24 external to the acoustic detectors 16, but disposed near and/or within a close proximity to the acoustic detectors 16. As noted above, each of the plurality of light emitting components 24 may be configured to provide sufficient light for the one or more acoustic detectors 16. Accordingly, in certain embodiments, it may be helpful to have a greater number of light emitting components 24 than acoustic detectors 16.

In certain embodiments, the element housing 18 may be a variety of shapes and/or configurations. For example, as illustrated in FIG. 2, the element housing 18 may be a rectangular and/or square configuration configured to house sensor components such as the acoustic detectors 16 and/or the light emitting components 24. In the illustrated embodiment of FIG. 3, the element housing is a circular element housing 72 that may be more compact than a square element housing 18 having the same number of sensor elements. Accordingly, in such embodiments, the acoustic elements 16 disposed in the arrangement 61 or 70 (e.g., the first acoustic detector 16a disposed adjacent to the second acoustic detector 16b, and having the third acoustic detector 16c disposed above and/or below the first acoustic detector 16a and/or the second acoustic detector 16b) may provide greater position tolerance while disposed in a compact circular element housing 18. Further, the element housing 18, the detectors 16, or the light emitting components 24 may be any other shape or configuration (e.g., triangular, pentagonal, octagonal, etc.) that may provide advantages in the amount of surface area or space occupied on the sensor body of the sensor 12. For example, an imaginary line connecting the light emitting components 24 may form a triangle. In certain embodiments, the separation gap 64 of the compact element housing 72 may be smaller than the separation gap 64 of the element housing 18, as illustrated in FIGS. 2-3. Accordingly, decreasing the separation gap 64 between the acoustic detectors 16 of the compact element housing 72 may allow the compact element housing 72 to decrease in size without drastically compromising the position tolerance benefits of the arrangements 61 and 70.

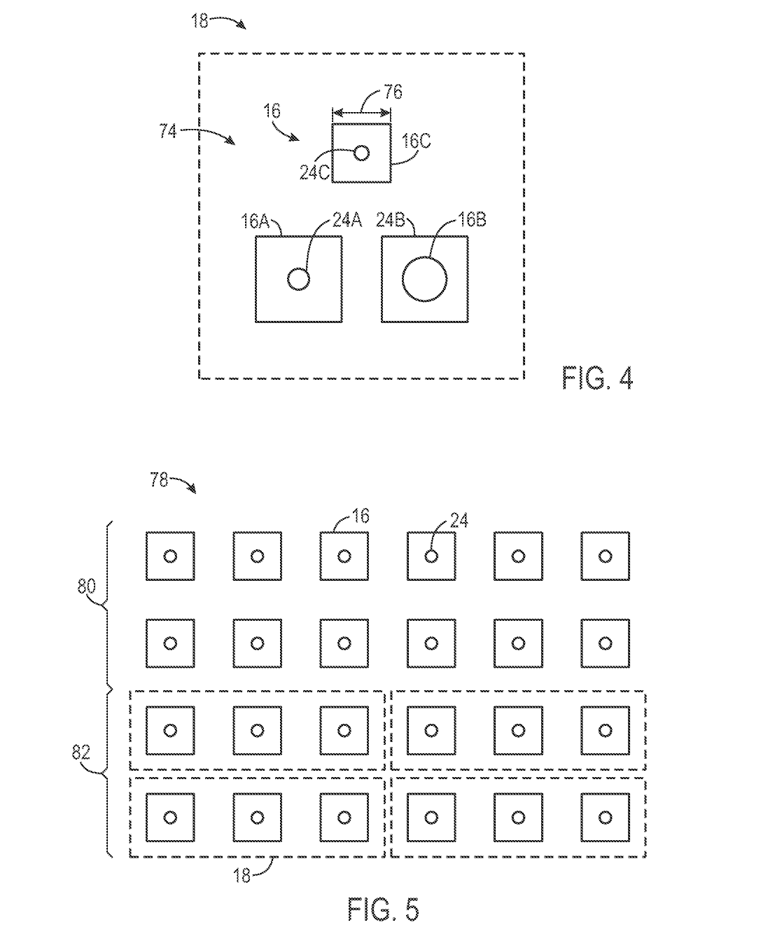

In certain embodiments, the sensor components disposed within the element housing 18 may be a variety of sizes and/or may have different proportions between sensor components, as further described with respect to FIG. 4, which illustrates an arrangement 74 (e.g., triangular arrangement 61) of the acoustic detectors 16 and the light emitting components 24 within the element housing 18, where each acoustic detector 16 and/or each light emitting component 24 may be a different size. Specifically, in embodiments where each light emitting component 24 is associated with a particular acoustic detector 16, the ratio between the amount of light emitted by the light emitting component 24 and the surface area of each acoustic detector 16 may vary. In certain embodiments, the ratio between the light emitting component 24 (e.g., the amount of the light emitted from the light emitting component 24, the size of the light emitting component 24, etc.) and the acoustic detector 26 (e.g., the surface area of the acoustic detector 16, the size of the acoustic detector 16, etc.) may be a optimal ratio, such that power is used in the most efficient but effective manner. For example, a first light emitting component 24a emits sufficient light to receive accurate and high quality measurements from the first acoustic detector 16a. In other embodiments, the ratio between the light emitting component 24 and the acoustic detector 26 may vary, such that a greater amount of light is provided from the light emitting component 24 (e.g., the second light emitting component 24b) than is optimal for the surface area of the acoustic detector 16 (e.g., the acoustic detector 16b). In certain embodiments, the optimal size ratio between the light emitting component 24 and the acoustic detector 16 may remain the same while the light emitting component 24 and the acoustic detector 16 may increase or decrease in size. For example, third acoustic detector 16c may be smaller than the first acoustic detector 16a and/or the second acoustic detector 16b, such that the third acoustic detector 16c is a third distance 76 in length. In certain embodiments, the third distance 76 may be smaller than the first distance 62 and/or the second distance 68.

FIG. 5 illustrates an embodiment of the one or more acoustic detectors 16 and the plurality of light emitting components 24 arranged in a two-dimensional array 78, where the array 78 may be disposed on the photoacoustic sensor 12 (e.g., patient-contacting surface of the photoacoustic sensor 12). In certain embodiments, the array 78 may be a plurality of acoustic detectors 16 disposed on the photoacoustic sensor 12 as a matrix of sensor elements 80. In other embodiments, the array 78 may be a plurality of element housings 18 disposed on the photoacoustic sensor 12 as a matrix of element housings 82, with each element housing 18 comprising one or more sensor elements. In the illustrated embodiment, the array 78 is a combination of the matrix of sensor elements 80 and the matrix of the element housing 82. Accordingly, the array 78 (e.g., matrix, ultrasound transducer array, transducer array, etc.) may include a plurality of acoustic detectors 16 and a plurality of light emitting components 24. The light emitting components 24 may be disposed via one or more optical fibers and/or light channels at approximately the center of the acoustic detector 16 and/or may be disposed external to the acoustic detectors 16. Further, the plurality of signals provided by the array 78 may be received and processed on separate paths or separate channels. For example, in certain embodiments, the array 78 may provide 8, 16, or 32 separate signals that may be processed on 8, 16, or 32 corresponding channels and/or paths, where each channel or path may include the switch 50, the amplifier 52, the filter 54, the A/D converter 56, and/or the QSM 58. In other embodiments, the array 78 may provide any number of separate signals (e.g., 9, 20, 24, 25, 28, 36, 49, etc.) that may be received and/or processed on corresponding channels and/or paths.

In certain embodiments, the signals received from the array 78 may be received through any number of separate channels, but may be processed and/or analyzed via the processor 38 with different signal processing techniques, such as, for example, beamforming (e.g., beamsteering) signal processing techniques. In such embodiments, the plurality of signals provided by the array 78 from the one or more acoustic detectors 16 and the light emitting components 24 may be used collectively (e.g., as one whole signal) for imaging techniques and/or additional signal processing. For example, the processor 38 may be configured to derive one or more physiological characteristics based on the total signal derived from the plurality of signals provided by the array 78. In other embodiments, the processor 38 may be configured to generate an image of the measurement site (e.g., whole tissue bed) for additional processing and/or may be configured to drive individual acoustic detectors 16, individual light emitting components 24, and/or individual element housings 18 to obtain more information from a particular portion or region of the array 78. Accordingly, beamforming signal processing techniques may be used to provide greater special selectivity on the measurement site of the subject's tissue. For example, the array 78 may be configured to gather information over a large measurement site, which may be further processed to select information from a particular portion of the measurement site, such as the portion of the measurement site having the target blood vessel.

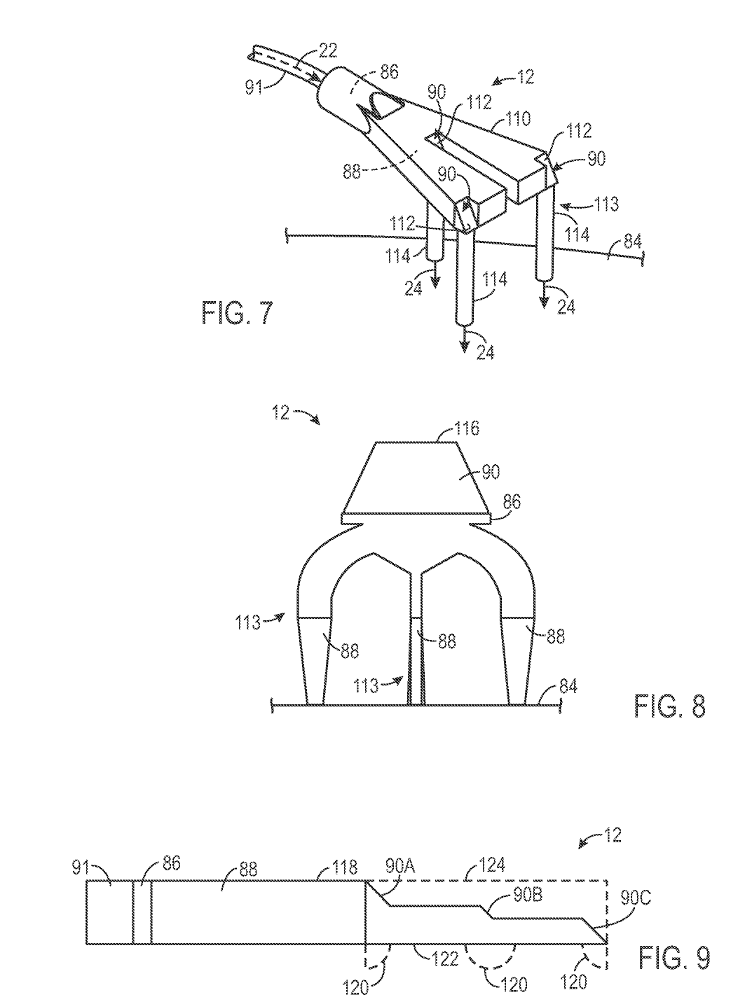

Accordingly, as noted above with respect to FIGS. 1-5, a plurality of acoustic detectors 16 and a plurality of light emitting components 24 may be spatially arranged on a sensor 12 to provide greater position tolerance, thereby helping to improve sensor 12 placement over the targeted blood vessels. In certain embodiments, it may be beneficial to provide light delivery methods and/or systems configured to function with the multi-element sensor 12 described in FIGS. 1-5. For example, in certain embodiments, the light delivery system 20 may be configured to receive the light source 22 parallel to the patient's tissue, split the light source 22 into a plurality of light emitting components 24, and provide the plurality of light emitting components 24 approximately normal to the tissue, such that each of the plurality of acoustic detectors 16 has a corresponding light emitting component 24, as described further with respect to FIG. 6. Further, in some embodiments, the light delivery methods and systems (e.g., components of the light delivery system 20, the detectors 16, the light emitting components 24, etc.) may be incorporated into a unitary (e.g., single-piece) housing structure, thereby helping to reduce the structural complexity of the multi-element sensor 12, as further described with respect to FIGS. 7-10B.

FIG. 6 illustrates an arrangement of the photoacoustic monitoring system 10 of FIG. 1 on a measurement site 84 on the patient in an indicator dilution application. Specifically, in the illustrated embodiment, the photoacoustic monitoring system 10 includes the patient monitor 14 and the photoacoustic sensor 12 having the light delivery system 20, the light source 22, the acoustic detectors 16, and the plurality of light emitting components 24. In certain embodiments, the light delivery system 20 may include a diffractive optical element 86 (e.g., DOE 86), an optical travel element 88 (e.g., OTE 88), and one or more prisms 90. In particular, the components of the light delivery system 20 may be configured to split the light source 22 into a plurality of distinct light emitting components 24 that are provided approximately normal (e.g., perpendicular, at a right angle) to the measurement site 84. Accordingly, the components of the light delivery system 20 may be positioned in a tangent plane to the surface of the measurement site 84.

The DOE 86 of the light delivery system 20 may be configured to receive the light source 22. As noted above with respect to FIG. 1, the light source 22 may be a single mode of light with a set of known optical properties. In certain embodiments, the patient monitor 14 may be configured to utilize the set of known optical properties to control the diffraction of the light source 22 into the plurality of distinct light emitting components 24 via the DOE 86. In other embodiments, the DOE 86 may be aligned with respect to the sensor 12, such that the plurality of light emitting components 24 generated from the light source 22 are positioned to target appropriate areas of interest on the measurement site 84. The DOE 86 may be a molded piece of plastic having the diffractive object embedded within, and the DOE 86 may be coupled to an optical fiber 91 associated with the light source 22. The OTE 88 of the light delivery system 20 may be configured to receive the plurality of light emitting components 24 split by the DOE 86. In certain embodiments, the OTE 88 is a spacer block configured to provide space for the plurality of light emitting components 24 to expand and/or separate into distinct light emitting components 24. The OTE 88 may be formed out of glass or plastic, and may be coated around the exterior to prevent the loss of light. The one or more light prisms 90 may be configured to receive the distinct plurality of light emitting components 24, and may be configured to deflect the light emitting components 24 such that they are normal to the measurement site 84. For example, the one or more prisms 90 may be configured to bend or angle the light emitting components 24 down towards the measurement site 84 by approximately 90 degrees.

It should be noted that in other embodiments, other configurations of the light delivery system 20 may be provided. For example, the DOE 86 may be disposed underneath the prism 90 and/or between the prism 90 and the acoustic detectors 16. In such embodiments, the DOE 86 thickness may be small to minimize the height of the sensor 12. Further, the acoustic detector 16 may be positioned directly underneath the prism 90, such that the plurality of light emitting components 24 are configured to travel through the acoustic detector 16 to the measurement site 84. For example, as noted above with respect to FIGS. 1, 2, 4, and 5, in certain embodiments, the plurality of light emitting components 24 may be configured to propagate through one or more holes or optical fibers disposed through the center of the acoustic detector 16. In other embodiments, such as within the illustrated embodiment, the plurality of light emitting components 24 may be provided to the measurement site 84 via holes or optical fibers disposed outside of the acoustic detector 16.

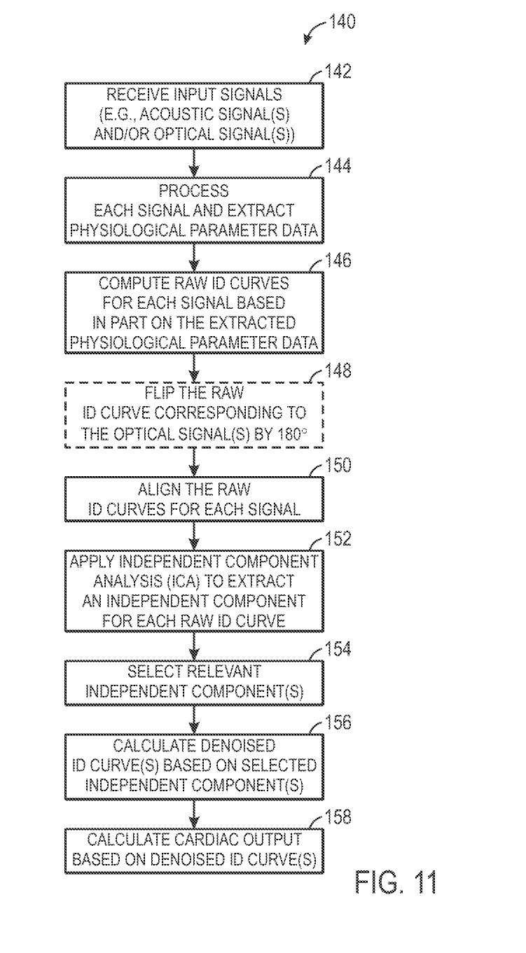

The light emitting components 24 may be provided to the measurement site 84 as photonic signals. Further, the blood in a blood vessel 92 and/or a constituent of the blood in the blood vessel 92 (such as the target blood vessel 92) such as, for example, hemoglobin or a bolus dose 93 (such as an injected indicator 93), may absorb at least some of the photonic signals at one or more monitoring sites (for example, a first monitoring site 94, a second monitoring site 96, or a third monitoring site 98). Accordingly, the blood may generate an acoustic pressure response via the photoacoustic effect, which may act on the surrounding tissues of the blood vessel 92. In the illustrated embodiment, the acoustic detector 16 disposed within the sensor 12 may be configured to detect and/or measure the acoustic pressure signals 100 traveling through the measurement site 84 (such as a tissue bed) of the subject. Changes in some properties of the blood in the blood vessel 92 at each of the monitoring sites may also be detected by the acoustic detector 16, such as changes that may occur in response to the bolus dose 93, a reduced hemoglobin concentration, etc. It should be noted that one or more acoustic detectors 16 (e.g., the acoustic detectors 16a, 16b, and 16c) may be configured to detect and/or measure the acoustic pressure signals 100 traveling through the measurement site 84. Accordingly, each acoustic detector 16 may be configured to detect and/or measure a different acoustic pressure signal 100 based on the acoustic pressure response generated at the monitoring sites 94, 96, or 98. The acoustic detectors 16 may be configured to output an acoustic pressure signal 102 to the patient monitor 14 for further processing via one or more paths. The patient monitor 14 may be configured to process the received acoustic signals 102 via one or more methods, as further explained with respect to FIGS. 11-13.

FIG. 7 is an embodiment of a light splitting structure 110 configured to house the components of the photoacoustic sensor 12, such as, for example, the light delivery system 20 and the one or more acoustic detectors 16. In particular, the structure 110 may be formed as a single unitary housing piece configured to house the components of the sensor 12. For example, in certain embodiments, the structure 110 may be formed via injection molding using one or more different types of injecting materials, such as, for example, thermoplastic or thermosetting polymers (e.g., polymethyl methacrylate (PMMA)), metals, glasses, elastomeres, confections, etc. The components of the light delivery system 20 (e.g., the DOE 86, the OTE 88, the prisms 90, the acoustic detectors 16, etc.) may be incorporated into and/or disposed within the light splitting structure 110. For example, in certain embodiments, the DOE 86 may be coupled to the optical fiber 91 and disposed within the structure 110, and may be configured to split the light source 22 into a plurality of light emitting components 24. Further, the distance within the structure 110 between the DOE 88 and the one or more prisms 90 may be configured to function as the OTE 88. In addition, in certain embodiments, the prisms 90 may be disposed within the structure 110 or incorporated into the structure 110. As noted above, the prisms 90 may be configured to bend the plurality of light emitting components 24 approximately 90 degrees. Accordingly, in certain embodiments, the prisms 90 may include a mirrored surface on a hypotenuse face of each prism 90 configured to redirect the light emitting components 24. In other embodiments, the prisms 90 may be entirely transparent, reflective, and/or may include two or more mirrors configured to redirect the light emitting components 24. In addition, the structure 110 may include one or more legs 113 configured to hold optical fibers 114 and/or light channels 114 (e.g., light pipes, light holes, etc.) having a reflective inner coating. Each of the optical fibers 114 may be associated with the light emitting component 24, and may be configured to aid in providing the light emitting components 24 normal the measurement site 84. In certain embodiments, each acoustic detector 16 may be incorporated, disposed within, and/or associated with the leg 113 of the structure 110.

FIG. 8 is a front view of an embodiment of an integrated prism light splitting structure 116 configured to house the components of the photoacoustic sensor 12 of FIG. 6. In particular, in the illustrated embodiment, the one or more prisms 90 of the light delivery system 90 may be integrated into the integrated prism structure 116 as a single prism 90. The integrated prism structure 116 may be formed via injection molding using one or more different types of injecting materials, such as, for example, thermoplastic or thermosetting polymers (e.g., polymethyl methacrylate (PMMA)), metals, glasses, elastomeres, confections, etc. Further, the single prism 90 may be configured to bend the plurality of light emitting components 24 or the light source 22 approximately 90 degrees, such that the light is provided normal to the measurement site 84. Accordingly, as noted above, the single prism 90 may include a mirrored surface and/or a mirrored coating on a hypotenuse face configured to redirect the light emitting components 24. In other embodiments, the single prism 90 may be entirely transparent, reflective, and/or may include two or more mirrors or mirrored coatings configured to redirect the light emitting components 24.

In certain embodiments, the prism 90 may be disposed above the DOE 86, such that the single source of light 22 angled normal to the measurement site 84 is then split into a plurality of light sources 86. It should be noted that in certain embodiments, the components of the light delivery system 20 may be configured in other arrangements within the structure (e.g., the housing 110 and/or the integrated prism housing 116). Further, the plurality of light emitting components 24 may each be associated with the leg 113 of the integrated prism structure 116. As noted above, each of the one or more legs 113 may be configured to hold the optical fibers 114 and/or the light channels 114 (e.g., light pipes, light holes, etc.) having a reflective inner coating, and may be configured to aid in providing the light emitting components 24 normal the measurement site 84. Further, each acoustic detector 16 may be incorporated, disposed within, and/or associated with the leg 113 of the structure 110.

FIG. 9 is a side view of an embodiment of a stepped-prism light splitting structure 118 configured to house the components of the photoacoustic sensor 12 of FIG. 6. In particular, in the illustrated embodiment, the stepped-prism structure 118 may include the one or more prisms 90 in a stepped and/or staggered arrangement along the length of the stepped-prism structure 118. The stepped-prism 118 may be configured to function with one or more acoustic detectors 16 arranged linearly, such as those depicted in FIG. 1. The stepped-prism 118 may be formed via injection molding using one or more different types of injecting materials, such as, for example, thermoplastic or thermosetting polymers (e.g., polymethyl methacrylate (PMMA)), metals, glasses, elastomeres, confections, etc. Further, the one or more prisms 90 within the stepped-prism 90 may be formed as mirrored surfaces 112 (not shown) and/or as mirrored coatings on the hypotenuse surface of each prism 90.

In the illustrated embodiment, the stepped-prism 118 includes three discrete prisms (e.g., a first prism 90a, a middle prism 90b, and a second prism 90c), where the middle prism 90b may be slightly smaller than the outer prisms (e.g., the first prism 90a and the third prism 90c). In such embodiments, the middle prism 90b may be smaller because the light density is higher in the center of the stepped-prism light splitting structure 90, and therefore a smaller area of light from the middle prism 90b is needed to be reflected down to the surface of the measurement site 84. In this manner, the amount of light reflected from each prism 90 within the stepped-prism light splitting structure 118 may be approximately uniform. For example, with a 2 mm optical fiber 91 configured to provide the light, the first prism 90a and the third prism 90c may be approximately 0.8 mm×0.8 mm×0.2 mm in size, while the middle prism 90b may be approximately 0.4 mm×0.4 mm×0.2 mm in size. Each of the prisms 90 within the stepped-prism 118 may be configured to provide a rectangular spot of light on the measurement surface 84. In certain embodiments, the stepped-prism 118 may include one or more lens 120 configured to provide circular spots of light on the measurement surface 84. It should be noted that while the illustrated embodiment depicts three prisms 90, in other embodiments, any number of prisms 90 may be utilized to provide light to the measurement site 84. In such embodiments, a smaller area of light from each of the middle prisms 90b may be reflected down to the surface of the measurement site 84. In addition, in certain embodiments, the thin stepped prism portion 122 of the stepped-prism light splitting structure 118 may be formed of a highly transmissive material configured for efficient light transmission and wave propagation. Further, the thin stepped prism portion 122 may be covered by a material 124 that is highly transmissive for wave propagation and may provide total internal reflection at each prism 90.

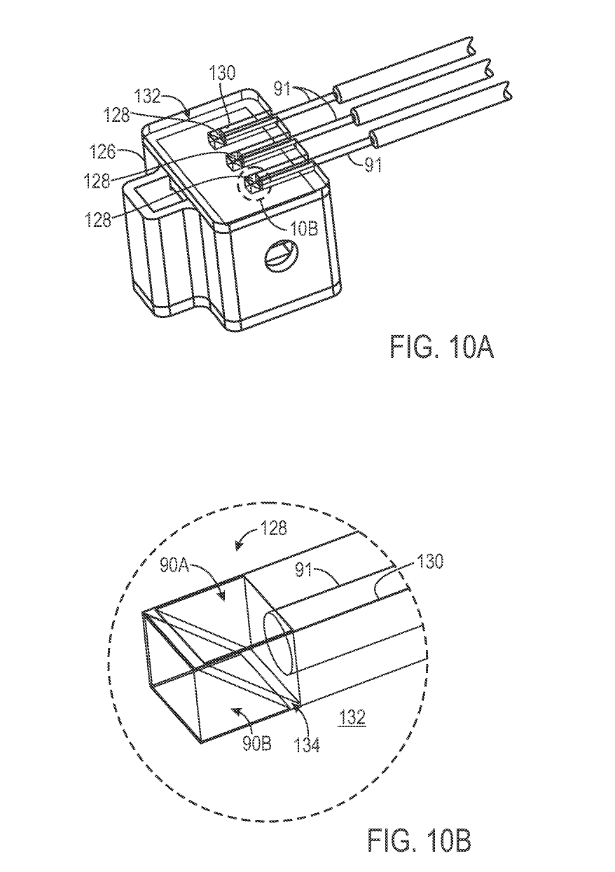

FIG. 10A is an embodiment of a multi-fiber light splitting structure 126 configured to receive a plurality of optical fibers 91 and to house the components of the photoacoustic sensor 12 of FIG. 6. As noted above, the optical fiber 91 may be associated with the light source 22, which may be single mode of light coupled to the light drive circuitry 48 within the patient monitor 14. In certain embodiments, a plurality of optical fibers 91 may be coupled to the light drive circuitry 48 and may be configured to provide a plurality of single light sources 22 (as depicted in FIG. 6) to the sensor 12. In such embodiments, the multi-fiber structure 126 may be configured to receive the plurality of optical fibers 91. In particular, each optical fiber 91 may be coupled to a prism subassembly 128, as further illustrated in FIG. 10B.

FIG. 10B is an embodiment of the prism subassembly 128 of the multi-fiber light splitting structure 126 of FIG. 10A illustrating two prisms 90a, 90b separated at the hypotenuse surface with an air gap 134. Generally, the hypotenuse surface of the prism 90a which is attached to the light delivering fiber 91 generates a large photoacoustic signal due to the high optical intensity on the reflective coating on that surface. This air gap 134 will greatly attenuate the photoacoustic signal and prevent it from propagating to the acoustic detector. The prism subassembly 128 may be configured to bend the light approximately 90 degrees, such that the light is provided normal to the measurement area 84.

In certain embodiments, each prism subassembly 128 of the multi-fiber structure 126 may be coupled to an optical fiber 91 and a rexolite cover 132 covering the multi-fiber structure 126. For example, the rexolite cover 132 of the multi-fiber structure 126 may include one or more slots 130 where the prism subassembly 128 may be securely disposed. Further, the prism subassembly 128 includes a prism 90a coupled to the tip of the optical fiber 91 and a prism 90b coupled to the rexolite cover of the multi-fiber structure 126. The two prisms 90a, 90b of the prism subassembly 128 may be separated by an air gap 134 along the hypotenuse surface of each prism 90. Accordingly, the two prisms 90a, 90b of the subassembly 128 may be secured to each of the optical fiber 91 and the rexolite cover 130 with adhesive on the outer edges of the prisms 90, while maintaining the air gap 134 between the two prisms 90. In an embodiment, the air gap 134 is approximately 0.1 mm.

FIGS. 1-10B describe various embodiments of systems and structures associated with the multi-element photoacoustic sensor 12 utilized within a photoacoustic system 10 configured for photoacoustic spectroscopy techniques. As noted above with respect to FIG. 1, the multi-element photoacoustic sensor 12 may be communicatively coupled to the patient monitor 14 within the photoacoustic system 10. Specifically, the patient monitor 14 may be configured to receive one or more acoustic pressure signals 102 based on the photoacoustic response of the tissue to the photonic signal 24. The acoustic pressure signal may be further processed by the patient monitor 14 to derive a photoacoustic signal, which in turn may be used to calculate any of a number of physiological parameters of interest, such as cardiac output. In particular, the patient monitor 14 may receive a plurality of acoustic pressure signals 102 and/or optical signals from the multi-element sensor 12. Accordingly, as described in detail with respect to FIGS. 11-13, it may be beneficial to provide methods to process and/or analyze the plurality of acoustic pressure signals 102 and/or optical signals provided by the multi-element sensor 12.

With the forgoing in mind, FIG. 11 is a flow chart of a method 140 for calculating cardiac output based on one or more denoised indicator dilution (ID) curves generated from the acoustic signals and/or the optical signals provided by the multi-element sensor 12 of FIGS. 1-10B. The method 140 begins with the patient monitor 14 receiving one or more input signals, such as acoustic pressure signals 102 or optical signals (block 142). The method further includes processing each received signal to extract a set of physiological parameter data via the processor 38 (block 144). For example, the processor 38 may calculate the amount or concentration of a constituent of interest in a localized region of tissue or blood using various algorithms based on the received acoustic pressure signal 102. Further, the processor 38 may be configured to compute a raw ID curve for each signal based at least in part on the extracted physiological parameter data from each input signal (block 146). For example, for each acoustic signal 102 or optical signal, the processor 38 may be configured to extract photoacoustic data or photoplethysmography data corresponding to the target blood vessel 92 from each monitoring site (e.g., 94, 96, and 98) at different times during the monitoring period. Based on the extracted data, the raw ID curve may be generated for each input signal. Accordingly, in multi-element sensors 12 having three acoustic detectors 16, three raw acoustic ID curves corresponding to each acoustic detector 16 and one raw optical ID curve corresponding to the optical detector 32 may be generated. In certain embodiments, the method 140 includes flipping the raw optical ID curve by approximately 180° (block 148), such that any time delays associated with the raw optical ID curve may be accounted for when time aligning each of the raw ID curves. After compensating for any time delays, the method 140 includes time aligning each of the raw ID curves (block 150).

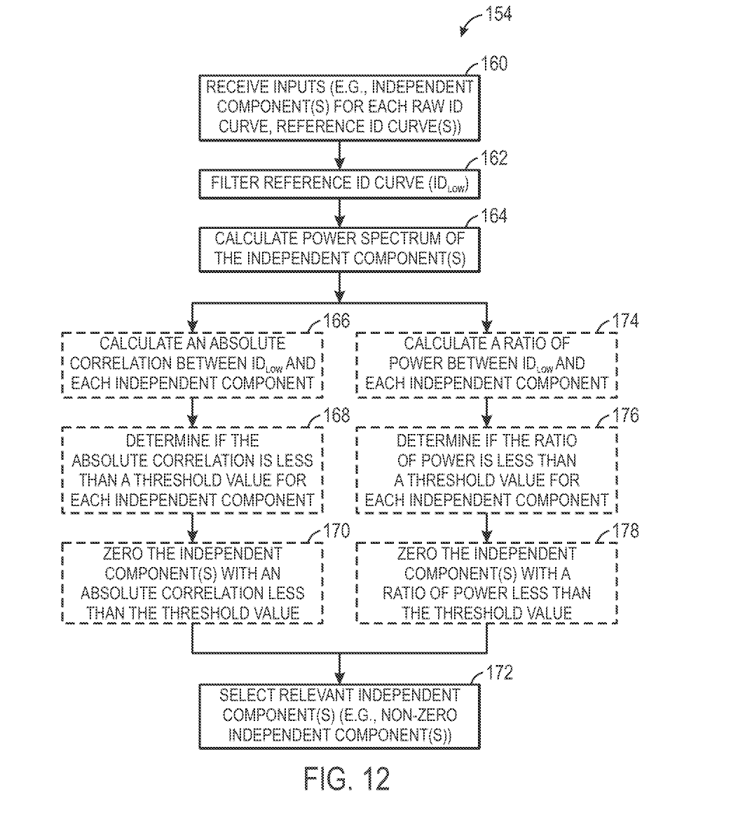

In particular, once aligned, the processor 38 may be configured to denoise each raw ID curve by applying one or more algorithms to the raw ID curve data, such as, for example, an independent component analysis (ICA) to each raw ID curve (block 152). Specifically, the ICA may be applied to extract an independent component for each raw ID curve. For example, types of independent components that may be determined for each raw ID curve may include noise sources (e.g., heart rate, cardiac noise, electronic interference noise, etc.) and the underlying ID signal. From the independent components determined for each raw ID curve, the relevant independent component (e.g., the underlying ID signal) may be selected, as will be further explained with respect to FIG. 12 (block 154). For example, in certain embodiments, for each raw ID curve, the relevant independent component corresponding to the signal may be selected among independent components relating to various noise sources. Accordingly, from four input signals (acoustic signals and/or optical signals), four relevant independent components corresponding to the underlying signal are determined via the ICA.

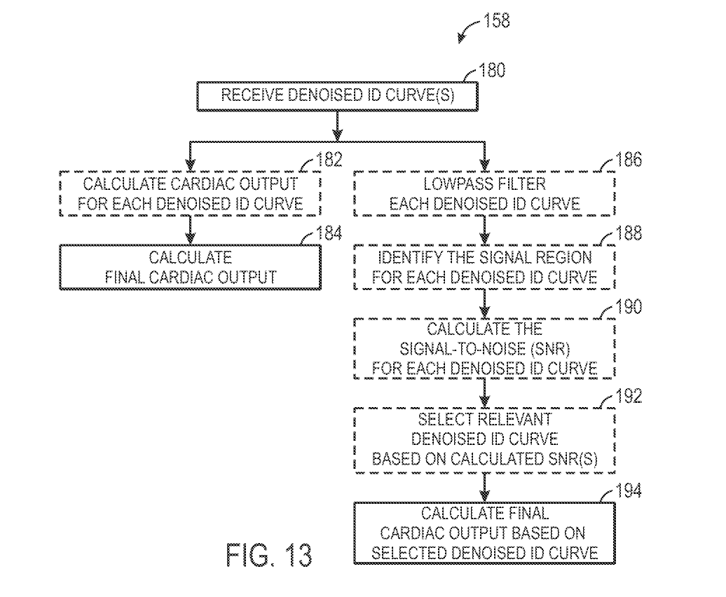

The method 140 may be further configured to calculate a denoised ID curve for each raw ID curve based on the selected relevant independent component for each raw ID curve (block 156). For example, in certain embodiments, the denoised ID curve for each raw ID curve may be calculated by inverse transforming the selected relevant independent components. In addition, the processor 38 may be configured to calculate a cardiac output based on the denoised ID curve (block 158). In certain embodiments, as further described in detail with respect to FIG. 13, the final cardiac output may be calculated based on the cardiac output calculated for each individual denoised ID curve, or the final cardiac output may be calculated from the most relevant denoised ID curve.

FIG. 12 is a flow chart of a method 154 for selecting relevant independent component(s) among the plurality of independent components generated for each raw ID curve, where each raw ID curve corresponds to the acoustic signal and/or the optical signal provided by the sensor 12 of FIGS. 1-10B. As noted above with respect to FIG. 11, the processor 38 may be configured to apply one or more algorithms, such as the ICA, to each raw ID curve generated for each input signal. Accordingly, for each raw ID curve, the ICA may generate one or more independent components corresponding to noise sources (e.g., heart rate, cardiac noise, electronic interference noise, etc.) and/or the underlying ID signal. In certain embodiments, it may be beneficial to determine the relevant independent components (e.g., the underlying ID signal) so that a denoised ID curve may be generated from the raw ID curve, as further explained below. Accordingly, the method 154 may be applied to each raw ID curve to determine the relevant independent components for each raw ID curve. In the illustrated embodiment, each raw acoustic ID curve is processed to determine the relevant independent components, however, it should be noted that in other embodiments, any signal received from the sensor 12 may be processed using similar techniques.

The method 154 may begin with receiving one or more inputs, such as the one or more independent components determined for each raw acoustic ID curve (block 160). As noted above, each independent component generated for each raw acoustic ID curve may be a relevant independent component (e.g., independent component corresponding to the underlying ID signal) or may be a non-relevant independent component (e.g., independent component corresponding to various noise sources). The method 154 further includes filtering the raw acoustic ID curve (e.g., ID_{low}), such as, for example, lowpass filtering the raw acoustic ID curve (block 162). Further, the method 154 includes calculating the power spectrum of each of the independent components for the given raw acoustic ID curve (block 164).

In some embodiments, selecting the relevant independent components may be determined by calculating an absolute correlation between the ID_{low} determined for the raw acoustic ID curve and each independent component corresponding to that raw acoustic ID curve, as determined through the ICA process (block 166). Further, in such embodiments, the processor 38 may be configured to determine if the absolute correlation calculated is less than a threshold value for each independent component (block 168). In particular, when the absolute correlation calculated is less than the threshold value for a particular independent component, the processor 38 may be configured to zero that independent component, so that it is no longer identified as a relevant independent component for that particular raw acoustic ID curve (block 170). Accordingly, in some embodiments, the relevant independent components for each raw acoustic ID curve may be determined by selecting the non-zero independent components determined based on the calculated absolute correlation between the ID_{low} corresponding to the raw acoustic ID curve and each independent component determined for that raw acoustic ID curve (block 172).

In some embodiments, selecting the relevant independent components may be determined by calculating a ratio of power between the power within the 0.2 Hz and the maximum power for the raw acoustic ID curve and each independent component corresponding to that raw acoustic ID curve, as determined through the ICA process (block 174). Further, in such embodiments, the processor 38 may be configured to determine if the ratio of power is less than a threshold value for each independent component (block 176). In particular, when the ratio of power calculated is less than the threshold value for a particular independent component, the processor 38 may be configured to zero that independent component, so that it is no longer identified as a relevant independent component for that particular raw acoustic ID curve (block 178). Accordingly, in some embodiments, the relevant independent components for each raw acoustic ID curve may be determined by selecting the non-zero independent components determined based on the calculated ratio of power between the power within the 0.2 Hz and the maximum power for the raw acoustic ID curve and each independent component corresponding to that raw acoustic ID curve (block 172).

Further, it should be noted that the method 154 may be repeated for each raw acoustic ID curve, so that at least one relevant independent component (e.g., corresponding to the underlying signal) may be determined for each raw acoustic ID curve. In certain embodiments, more than one relevant independent component may be determined for each raw acoustic ID curve. In such embodiments, the processor 38 may be configured to calculate two or more denoised ID curves based on each selected independent component, as explained in detail with respect to FIG. 11 (block 156).

FIG. 13 is a flow chart for calculating the final cardiac output based on the one or more denoised ID curves of FIG. 11. As noted above with respect to FIG. 11, the method 140 may be utilized to calculate a denoised ID curve for each raw ID curve based on the selected relevant independent component for each raw ID curve (block 156). For example, each denoised ID curve may correspond to the denoised acoustic pressure signal 102 received from a single acoustic detector 16 among the plurality of acoustic detectors 16 on the multi-element sensor 12. Accordingly, in certain embodiments, it may be beneficial to determine a final cardiac output based on a single acoustic pressure signal 102 received from the sensor 12, where the single acoustic pressure signal 102 is determined to be the more relevant signal 102 received from the multi-element sensor 12. In other embodiments, it may be beneficial to determine a final cardiac output based on all of the acoustic pressure signals 102 received from the sensor 12, where all the acoustic pressure signals are determined to be relevant signals 102 received from the multi-element sensor 12.

With the forgoing in mind, the method 158 begins with receiving one or more denoised ID curves, where each denoised ID curve may correspond to the denoised acoustic pressure signal received from the acoustic detector 16 of the multi-element sensor 12 (block 180). In certain embodiments, the method 158 includes calculating a cardiac output for each denoised ID curve determined (block 182). In particular, the final cardiac output may be a mean, a median, and/or a weighted combination of each cardiac output for each denoised ID curve determined (block 184).

In other embodiments, the method 158 includes selecting one or more relevant denoised ID curves among the available denoised ID curves. For example, the method 158 includes lowpass filtering each denoised ID curve (block 186) and identifying the signal region for each denoised ID curve (block 188). Further, the method 158 includes calculating a signal-to-noise ratio (SNR) for each denoised ID curve (block 190). Based on the calculated SNR for each denoised ID curve, the processor 38 may be configured to select one or more relevant denoised ID curves (block 192). In particular, the final cardiac output may be calculated based on the selected one or more denoised ID curves (block 194).

While the disclosure may be susceptible to various modifications and alternative forms, specific embodiments have been shown by way of example in the drawings and have been described in detail herein. Further, specific elements of the disclosed embodiments may be combined or exchanged with one another. It should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A photoacoustic sensor, comprising:
a plurality of light emitters coupled to a light source, wherein each respective light emitter emits one or more wavelengths of light into a tissue region of a patient; and
a plurality of acoustic detectors disposed on a tissue-contacting surface of the photoacoustic sensor, and wherein one or more of the plurality of acoustic detectors is disposed to detect acoustic energy generated by the tissue region of the patient in response to the emitted light.

2. The photoacoustic sensor of claim 1, wherein the light source comprises one or more light emitting diodes, one or more laser diodes, a pulsed laser, a continuous wave laser, or a vertical cavity surface emitting laser.

3. The photoacoustic sensor of claim 1, wherein the plurality of acoustic detectors comprises an ultrasound transducer.

4. The photoacoustic sensor of claim 1, wherein each of the plurality of light emitters passes through a channel or opening within each of the respective plurality of acoustic detectors such that a portion of each of the light emitters is surrounded by its respective acoustic detector.

5. The photoacoustic sensor of claim 4, wherein at least a portion of the plurality of acoustic detectors is arranged in a triangular configuration such that the light emitters form vertices of an imaginary triangle.

6. The photoacoustic sensor of claim 1, wherein the plurality of acoustic detectors and the plurality of light emitters are disposed on the tissue-contacting surface in a linear array.

7. The photoacoustic sensor of claim 1, wherein the plurality of acoustic detectors are spaced apart from one another on the tissue-contacting surface to form gaps between adjacent acoustic detectors and wherein the plurality of light emitters are positioned within the gaps.

8. The photoacoustic sensor of claim 1, comprising a light splitter comprising:
a source end configured to receive the light source at an angle less than normal to the tissue;
a light prism within the structure configured to bend the light towards the tissue region; and
one or more tissue-contacting ends configured to contact the tissue and direct the light into the tissue.

9. The photoacoustic sensor of claim 8, wherein the light splitter comprises a diffracting optical element configured to split the light into a plurality of light beams directed into respective tissue-contacting ends.

10. The photoacoustic sensor of claim 8, wherein the light prism is stepped and wherein the acoustic detectors are arranged linearly along the stepped prism.

11. The photoacoustic sensor of claim 10, wherein individual steps of the stepped prism are different sizes such that light reflected from the prism is uniform.

12. A photoacoustic system, comprising:
a sensor input; and
a patient monitor communicatively coupled to the sensor input and configured to:
drive one or more light sources to provide light to the plurality of light emitters;
receive a signal from the plurality of acoustic detectors, wherein the signal corresponds to the acoustic energy detected by one or more of the plurality of acoustic detectors;
determine a quality of the signal associated with driving all or only a portion of the light emitters simultaneously; and
determine a physiological parameter value based on a highest quality signal.

13. The photoacoustic system of claim 12, wherein the patient monitor is configured to drive only a portion of the light emitters when the signal quality associated with driving only the portion is above a threshold.

14. A method, comprising:
using a processor to perform the steps of:
receiving an acoustic pressure signal from an acoustic detector disposed on a multi-element photoacoustic sensor, wherein the multi-element photoacoustic sensor comprises two or more acoustic detectors.
generating a raw indicator dilution curve for the acoustic pressure signal based at least in part on physiological parameter data extracted from the acoustic pressure signal;
applying an independent component analysis algorithm to extract one or more independent components corresponding to the raw indicator dilution curve;
selecting one or more relevant independent components from among the one or more extracted independent components, wherein the relevant independent component corresponds to the underlying signal of the raw indicator dilution curve;
calculating one or more denoised indicator dilution curves based at least in part on the one or more relevant independent components selected; and
calculating a final cardiac output based at least on the one or more denoised indicator dilution curves.

15. The method of claim 14, wherein selecting the one or more relevant independent components comprises determining one or more non-relevant independent components corresponding to the raw indicator dilution curve.

16. The method of claim 14, wherein the non-relevant independent components comprise a heart rate noise, a cardiac noise, an electronic interference noise, or a combination thereof.

17. The method of claim 14, wherein selecting the one or more relevant independent components comprises calculating a power spectrum for each independent component.

18. The method of claim 17, wherein selecting the one or more relevant independent components comprises determining if an absolute correlation between the raw indicator curve and each independent component is less than a threshold value for each independent component.

19. The method of claim 17, wherein selecting the one or more relevant independent components comprises determining if a ratio of power between the raw indicator curve and each independent component is less than a threshold value for each independent component.

20. The method of claim 14, wherein calculating the final cardiac output comprises selecting a relevant denoised indicator dilution curve and calculating the final cardiac output based at least in part on the selected relevant denoised indicator dilution curve.
